(19) United States
(12) Patent Application Publication
(10) Pub. No.: US 2017/0007507 A1
(43) Pub. Date: Jan. 12, 2017

(54) FREE STANDING COLOR COSMETIC STICK

(71) Applicant: COTY INC., New York, NY (US)

(72) Inventors: Salvatore J. Barone, Staten Island, NY (US); Irina Staina, Branchburg, NJ (US)

(21) Appl. No.: 15/116,475

(22) PCT Filed: Feb. 4, 2015
(86) PCT No.: PCT/US2015/014428

§ 371 Date: Aug. 3, 2016

Related U.S. Application Data
(60) Provisional application No. 61/937,163, filed on Feb. 7, 2014.

(51) Int. Cl. A61K 8/02; A61K 8/92; A61K 8/97; A61Q 1/10; A61K 8/26; A61K 8/25; A61Q 1/02; A61Q 1/08; A61K 8/37; A61K 8/891

(52) U.S. Cl. CPC A61K 8/0229; A61K 8/37; A61K 8/92; A61K 8/922; A61K 8/97; A61K 8/891; A61K 8/26; A61K 8/25; A61Q 1/02; A61Q 1/08; A61Q 1/10; A61K 2800/30

(57) ABSTRACT

Inventive embodiments disclosed herein includes a free standing cosmetic stick. The free standing stick includes a formulation that includes Isopentyl laurate in a concentration of at least 33% by weight. The formulation also includes one or more coloring agents; one or more constituents for rendering the formulation into a solid stick; wherein the formulation is free from volatile ingredients.

### CLAIM OF PRIORITY

This Application claims the benefit of priority to U.S. Provisional Patent Application No. 61/937,163, filed Feb. 7, 2014, which is incorporated herein by reference in its entirety.

### FIELD

Inventive subject matter disclosed herein relates to a free standing color cosmetic stick for applying color. The free standing stick is transfer resistant and contains no volatile ingredients.

### BACKGROUND

The first color stick was lipstick. Elizabeth Arden and Estee Lauder began selling tube-shaped lipstick in the 1920′s. Carnauba wax has traditionally provided the structure for lipstick. Lipstick has also included fillers such as silica and has few, if any emollients.

### SUMMARY

Inventive embodiments disclosed herein include a free standing color cosmetic stick. The free standing color cosmetic stick includes a formulation having isopentyl laurate in a concentration of at least 33% by weight. The free standing stick also includes one or more coloring agents and one or more constituents for rendering the formulation into a solid stick. The free standing color cosmetic stick is free from volatile ingredients.

Another embodiment includes a free standing color cosmetic stick for imparting a foundation to a user's skin. The free standing foundation cosmetic stick includes isopentyl laurate in a concentration of at least 33% by weight; petrolatum; Carnauba wax; Euphorbia cerifera; dimethicone/divinyldimethicone/silsesquioxane crosspolymer and Copernicia Cerifera wax; one or more color agents; and polymethylsilsesquioxane.

Another embodiment of the free standing color cosmetic stick includes a free standing cosmetic stick for imparting a blush to a user's skin. The free standing blush cosmetic stick includes isopentyl laurate in a concentration of at least 33% by weight; microcrystalline wax; Ozokerite; Carnauba wax; Candelilla wax; as well as

Dimethicone/divinyldimethicone/silsesquioxane crosspolymer and Copernicia Cerifera wax; kaolin; talc and polymethylsilsesquioxane.

Another embodiment of the free standing color cosmetic stick includes a free standing cosmetic for imparting an eyeshadow to a user's skin. The free standing eyeshadow stick includes isopentyl laurate in a concentration of at least 33% by weight; petrolatum; microcrystalline wax; Carnauba wax; Candelilla wax, dimethicone/divinyldimethicone/silsesquioxane crosspolymer and Copernicia Cerifera wax; as well as silica; kaolin; and talc

One other embodiment includes a free standing color cosmetic stick having isopentyl laurate in a concentration of at least 33% by weight and one or more agents for imparting color.

Another embodiment includes a method of making a free standing color cosmetic stick for imparting color to an end user. The method includes preparing a formulation comprising isopentyl laurate in a concentration of at least 33% by weight and one or more agents for imparting color; heating the formulation and forming the formulation into a stick shape to make a formed stick shape; and cooling the formed stick.

### DESCRIPTION OF THE DRAWINGS

FIG. 1 illustrates an embodiment for a free standing foundation stick.

FIG. 2 illustrates an embodiment for a free standing blush stick.

FIG. 3 illustrates an embodiment for a free standing eye shadow stick.

### DETAILED DESCRIPTION

Embodiments of the invention are described in sufficient detail to enable those skilled in the art to practice the invention. Other embodiments may be utilized and structural, logical, chemical, and other changes may be made without departing from the spirit or scope of the invention discussed herein. The following detailed description is, therefore, not to be taken in a limiting sense, and the scope of the invention discussed herein is defined only by the appended claims.

Inventive embodiments disclosed herein are directed to a free standing color cosmetic stick for imparting color to an end user. The free standing color cosmetic stick includes isopentyl laurate in a concentration of at least 33% by weight. The free standing color cosmetic stick also includes one or more coloring agent and one or more constituents for rendering a formulation into a solid stick. The free standing stick is free from volatile ingredients.

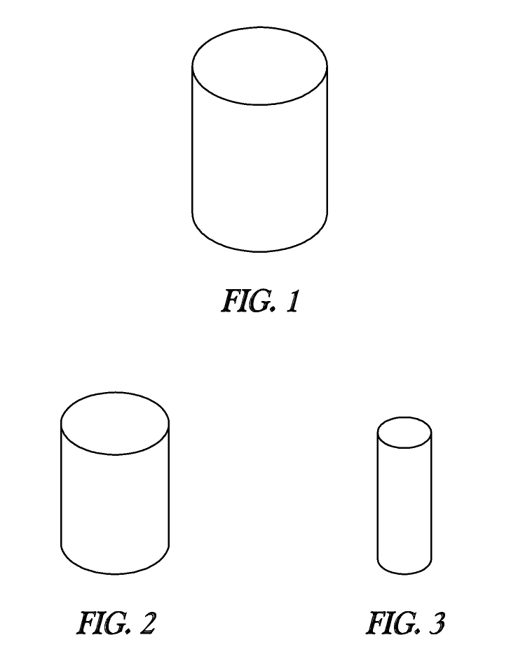

Embodiments of the free standing cosmetic stick are shown in FIGS. 1, 2, and 3 for foundation, blush and eye shadow, respectively. The term “free standing stick” refers to stick configurations such as are shown in FIGS. 1,2 and 3.

The free standing color cosmetic stick imparts a color to a user's skin that is transfer resistant and that contains no volatile components. The formulation of the free-standing cosmetic stick includes the natural and biodegradable material, isoamyl (isopentyl) laurate. Isoamyl laurate is a multifunctional emollient, which provides an excellent silicon feeling and a good skin compatibility. The chemical structure for isoamyl laurate is as follows:

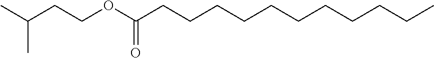 [SMILES: CCCCCCCCCCCC(=O)OCCC(C)C]

Embodiments of the free standing color stick include formulations having one or more of petrolatum, microcrystalline wax and ozokerite, depending upon the particular application.

Petrolatum is also known as perfecta; saxoline; VASELINE; PARAFFIN; PARAFIN; cosmoline; FEMA 3216; WAX WHITE; PARAFFINE. Petrolatum has a CAS No. of 8009-03-8. Petrolatum is a purified mixture of semisolid saturated hydrocarbons having the general formula C(n)H(2n+2).

Microcrystalline, as used herein, refers to “microcrystalline wax.” The microcrystalline wax is a hardening wax and has a melting point ranging from 85 to 95 degrees Centigrade. The hardness ranges from 6 to 14 decimillimeters (dmm) The microcrystalline wax is added to the free standing cosmetic stick formulation as a granule, pellet, slab or liquid bulk.

Ozokerite wax is a naturally occurring, light yellow to dark brown mineral wax composed of solid paraffinic hydrocarbons.

For a color free standing stick used as a foundation, petrolatum ranges in concentration from 5-6 percent by weight. The microcrystalline wax ranges in concentration from 2 to 3 percent by weight. Ozokerite ranges in concentration from 1 to 2 percent by weight. A color stick used as a blush has a microcrystalline concentration of 4 to 5 percent by weight and a ozokerite concentration of 3 to 4 percent by weight. The blush formulation is free from petrolatum. An eye shadow free standing stick includes a microcrystalline concentration of 2 to 3 percent by weight; an ozokerite concentration of 1 to 2 percent by weight; and a petrolatum concentration of 5 to 6 percent by weight

The free standing cosmetic stick embodiments also include Carnauba wax and Candelilla wax in concentrations that total about 4 to 6 percent by weight of the free standing stick formulation. A formulation that includes dimethicone/divinyl dimethicone/Silsesquioxane crossploymer; Copernecia and Cerafera wax is added to the foundation and blush formulation embodiments in a concentration of 3 to 4 percent by weight to impart an effective degree of transfer to a user's skin. The dimethicone/divinyl dimethicone/silsesquioxane crossploymer; Copernecia Cerafera wax mixture is added in a concentration of 4 to 5 percent by weight for an eye shadow formulation.

The free standing cosmetic stick formulation includes polyglycerol-3 diisostearate in a concentration of 0.2 to 0.3 percent by weight. The polyglycerol-3 diisostearate acts as a thickener and an emulsifier. Tocopherol is added in a concentration of 0.1 percent by weight.

Ingredients to impart color and sparkle to the free standing stick include silica, kaolin, talc, mica, titanium dioxide, iron oxide, black oxide, and polymethylsisesquioxane.

The free standing color cosmetic stick is made with a hop pour technique developed to formulate free standing bullets without any volatile ingredients.

Specific examples of the free standing cosmetic stick embodiments are as follows and are presented to exemplify inventive embodiments but not limit them.

### EXAMPLE 1

### A Free Standing Foundation

| Constituent | % w/w |
|---|---|
| Isopentyl laurate | 33.10 |
| Petrolatum | 5.30 |
| Macrocrystalline | 2.80 |
| Ozokerite | 1.50 |
| Copernica Cerifers (Carnauba) Wax | 2.20 |
| Euphorbia cerifera (Candalilla wax) | 2.00 |
| Dimethicone/Divinyl dimethicone/Silsesquioxane | 4.50 |
| crossploymer; Copernecia Cerafera | |
| Polyglycerol-3 Diisostereate | 0.25 |
| Silica | 0.50 |
| Kaolin | 13.30 |
| Talc | 17.00 |
| Mica | 4.50 |
| Caprylyl Glycol | 0.50 |
| Titanium Dioxide | 8.00 |
| Iron Oxide | 1.31 |
| Iron oxide | 0.44 |
| Black Oxide | 0.22 |
| Polymethylsisesquioxane | 2.50 |
| Tocopherol | 0.10 |
| Total | 100.00 |

### EXAMPLE 2

### Free Standing Blush

| Constituent | % w/w |
|---|---|
| Isopentyl laurate | 34.2 |
| Petrolatum | 0.00 |
| Macrocrystalline | 4.80 |
| Ozokerite | 3.50 |
| Copernica Cerifers (Carnauba) Wax | 2.20 |
| Euphorbia cerifera (Candalilla wax) | 1.60 |
| Dimethicone/Divinyl dimethicone/Silsesquioxane | 3.50 |
| crossploymer; Copernecia Cerafera wax | |
| Polyglycerol-3 Diisostereate | 0.50 |
| Caprilic/Capric Triglyceride, Stearalkonium Hectorite, | 0.50 |
| Propylene Carbonate | |
| Silica | 0.50 |
| Kaolin | 12.80 |
| Talc | 24.30 |
| Mica | 5.50 |
| Caprylyl Glycol | 0.50 |
| Titanium Dioxide | 1.61 |
| Iron Oxide | 0.00 |
| Iron oxide | 0.63 |
| Black Oxide | 0.22 |
| Polymethylsisesquioxane | 2.00 |
| Tocopherol | 0.10 |
| Total | 100.00 |

### EXAMPLE 3

### Free Standing Eyeshadow

| Constituent | % w/w |
|---|---|
| Isopentyl laurate | 33.10 |
| Petrolatum | 5.30 |
| Macrocrystalline | 2.80 |
| Ozokerite | 1.50 |
| Copernica Cerifers (Carnauba) Wax | 2.20 |
| Euphorbia cerifera (Candalilla wax) | 2.00 |
| Dimethicone/Divinyl dimethicone/Silsesquioxane | 4.50 |
| crossploymer; Copernecia Cerafera wax | |
| Polyglycerol-3 Diisostereate | 0.25 |
| Caprilic/Capric Triglyceride, Stearalkonium Hectorite, | 0.00 |
| Propylene Carbonate | |
| Silica | 0.50 |
| Kaolin | 11.00 |
| Talc | 17.00 |
| Mica | 1.00 |
| Caprylyl Glycol | 0.50 |
| Titanium Dioxide | 7.00 |
| Iron Oxide | 0.00 |
| Iron oxide | 0.63 |
| Black Oxide | 0.65 |
| Polymethylsisesquioxane | 2.50 |
| Tocopherol | 0.10 |
| Total | 100.00 |

Although specific embodiments have been illustrated and described herein, it will be appreciated by those of ordinary skill in the art that any procedure that is calculated to achieve the same purpose may be substituted for the specific embodiments shown. This application is intended to cover any adaptations or variations of the present subject matter.

What is claimed is:

1. A free standing cosmetic stick, comprising:
a formulation comprising isopentyl laurate in a concentration of at least 33% by weight;
one or more coloring agents;
one or more constituents for rendering the formulation into a solid stick;
wherein the formulation is free from volatile ingredients.

2. A free standing cosmetic stick, comprising:
isopentyl laurate in a concentration of at least 33% by weight;
petrolatum;
carnauba wax;
euphorbia cerifera;
dimethicone/divinyldimethicone/silsesquioxane crosspolymer and Copernicia Cerifera wax;
color agent; and
polymethylsilsesquioxane.

3. A free standing cosmetic stick, comprising:
isopentyl laurate in a concentration of at least 33% by weight;
microchrystalline;
ozokerite;
carnauba wax;
candelilla wax;
dimethicone/divinyldimethicone/silsesquioxane crosspolymer and Copernicia Cerifera wax;
kaolin;
talc; and
polymethylsilsesquioxane.

4. A free standing cosmetic stick, comprising:
isopentyl laurate in a concentration of at least 33% by weight;
petrolatum;
microcrystalline;
carnauba wax;
candelilla wax;
dimethicone/divinyldimethicone/silsesquioxane crosspolymer and Copernicia Cerifera wax;
silica;
kaolin; and
talc.

5. A free standing color cosmetic stick comprising isopentyl laurate in a concentration of at least 33% by weight and one or more agents for imparting color.

6. The free standing stick of claim 5, further comprising one or more constituents for rendering the formulation into a solid stick;

7. The free standing stick of claim 5, wherein the free standing color cosmetic stick is free from volatile ingredients.

8. A method of making a free standing cosmetic stick for imparting color to an end user, comprising:
preparing a formulation comprising isopentyl laurate in a concentration of at least 33% by weight and one or more agents for imparting color;
heating the formulation and forming the formulation into a stick shape to make a formed stick shape; and
cooling the formed stick.

9. The method of claim 8, wherein the free standing cosmetic stick is formed in a mold.
